(19) United States
(12) Patent Application Publication
(10) Pub. No.: US 2022/0401740 A1
(43) Pub. Date: Dec. 22, 2022

(54) Ramping of Neural Dosing for Comprehensive Spinal Cord Stimulation Therapy

(71) Applicant: Boston Scientific Neuromodulation Corporation, Valencia, CA (US)

(72) Inventor: Travis McCoy, Slidell, LA (US)

(21) Appl. No.: 17/806,652

(22) Filed: Jun. 13, 2022

Related U.S. Application Data
(60) Provisional application No. 63/211,875, filed on Jun. 17, 2021.

(51) Int. Cl. A61N 1/36; A61N 1/05

(52) U.S. Cl. CPC A61N 1/36157; A61N 1/0551; A61N 1/36062; A61N 1/36175

(57) ABSTRACT

Methods and systems for providing sub-perception spinal cord stimulation are described. In some examples, the stimulation current is shared among three or more anodes and three or more cathodes to provide virtual poles that are configured to cover a relatively large area of the patient's neural tissue that contains the “sweet spot” for treating the patient's pain. Covering a relatively large area mitigates the need to perform time-intensive sweet spot searching. In some examples, one or more stimulation parameters are varied while the stimulation is being provided.

### CROSS REFERENCE TO RELATED APPLICATIONS

This application is a non-provisional of U.S. Provisional Patent Application Ser. No. 63/211,875, filed Jun. 17, 2021, which is incorporated herein by reference in its entirety, and to which priority is claimed.

### FIELD OF THE INVENTION

This application relates to Implantable Medical Devices (IMDs), generally, Spinal Cord Stimulators, more specifically, and to methods of control of such devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system comprising a spinal cord stimulator, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible device case 12 that holds the circuitry and battery 14 necessary for the IPG to function. The IPG 10 is coupled to electrodes 16 via one or more electrode leads 15 that form an electrode array 17. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to circuitry within the case 12, although these details aren't shown.

In the illustrated IPG 10, there are sixteen lead electrodes (E1-E16) split between two leads 15, with the header 23 containing a 2×1 array of lead connectors 24. However, the number of leads and electrodes in an IPG is application specific and therefore can vary. The conductive case 12 can also comprise an electrode (Ec). In a SCS application, the electrode leads 15 are typically implanted proximate to the dura in a patient's spinal column on the right and left sides of the spinal cord midline. The proximal electrodes 22 are tunneled through the patient's tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient's tissue. The IPG leads 15 can be integrated with and permanently connected the case 12 in other IPG solutions. The goal of SCS therapy is to provide electrical stimulation from the electrodes 16 to alleviate a patient's symptoms, most notably chronic back pain.

IPG 10 can include an antenna 26a allowing it to communicate bi-directionally with a number of external devices, as shown in FIG. 4. The antenna 26a as depicted in FIG. 1 is shown as a conductive coil within the case 12, although the coil antenna 26a can also appear in the header 23. When antenna 26a is configured as a coil, communication with external devices preferably occurs using near-field magnetic induction. IPG may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves. RF antenna 26b may operate in accordance with any number of known RF communication standards, such as Bluetooth, Zigbee, WiFi, MICS, and the like.

Stimulation in IPG 10 is typically provided by pulses, as shown in FIG. 2. Stimulation parameters typically include the amplitude of the pulses (A; whether current or voltage); the frequency (F) and pulse width (PW) of the pulses; the electrodes 16 (E) activated to provide such stimulation; and the polarity (P) of such active electrodes, i.e., whether active electrodes are to act as anodes (that source current to the tissue) or cathodes (that sink current from the tissue). These stimulation parameters taken together comprise a stimulation program that the IPG 10 can execute to provide therapeutic stimulation to a patient.

In the example of FIG. 2, electrode E5 has been selected as an anode, and thus provides pulses which source a positive current of amplitude +A to the tissue. Electrode E4 has been selected as a cathode, and thus provides pulses which sink a corresponding negative current of amplitude −A from the tissue. This is an example of bipolar stimulation, in which only two lead-based electrodes are used to provide stimulation to the tissue (one anode, one cathode). However, more than one electrode may act as an anode at a given time, and more than one electrode may act as a cathode at a given time (e.g., tripole stimulation, quadripole stimulation, etc.).

The pulses as shown in FIG. 2 are biphasic, comprising a first phase 30a, followed quickly thereafter by a second phase 30b of opposite polarity. As is known, use of a biphasic pulse is useful in active charge recovery. For example, each electrodes' current path to the tissue may include a serially-connected DC-blocking capacitor, see, e.g., U.S. Patent Application Publication 2016/0144183, which will charge during the first phase 30a and discharged (be recovered) during the second phase 30b. In the example shown, the first and second phases 30a and 30b have the same duration and amplitude (although opposite polarities), which ensures the same amount of charge during both phases. However, the second phase 30b may also be charged balance with the first phase 30a if the integral of the amplitude and durations of the two phases are equal in magnitude, as is well known. The width of each pulse, PW, is defined here as the duration of first pulse phase 30a, although pulse width could also refer to the total duration of the first and second pulse phases 30a and 30b as well. Note that an interphase period (IP) during which no stimulation is provided may be provided between the two phases 30a and 30b.

IPG 10 includes stimulation circuitry 28 that can be programmed to produce the stimulation pulses at the electrodes as defined by the stimulation program. Stimulation circuitry 28 can for example comprise the circuitry described in U.S. Provisional Patent Application Ser. Nos. 62/386,000 and 62/393,003, both filed Sep. 10, 2016, or described in U.S. Pat. Nos. 8,606,362 and 8,620,436. These references are incorporated herein by reference.

FIG. 3 shows an external trial stimulation environment that may precede implantation of an IPG 10 in a patient. During external trial stimulation, stimulation can be tried on a prospective implant patient without going so far as to implant the IPG 10. Instead, one or more trial leads 15′ are implanted in the patient's tissue 32 at a target location 34, such as within the spinal column as explained earlier. The proximal ends of the trial lead(s) 15′ exit an incision 36 and are connected to an External Trial Stimulator (ETS) 40. The ETS 40 generally mimics operation of the IPG 10, and thus can provide stimulation pulses to the patient's tissue as explained above. See, e.g., U.S. Pat. No. 9,259,574, disclosing a design for an ETS. The ETS 40 is generally worn externally by the patient for a short while (e.g., two weeks), which allows the patient and his clinician to experiment with different stimulation parameters to try and find a stimulation program that alleviates the patient's symptoms (e.g., pain). If external trial stimulation proves successful, trial lead(s) 15′ are explanted, and a full IPG 10 and lead(s) 15 are implanted as described above; if unsuccessful, the trial lead(s) 15′ are simply explanted.

Like the IPG 10, the ETS 40 can include one or more antennas to enable bi-directional communications with external devices, explained further with respect to FIG. 4. Such antennas can include a near-field magnetic-induction coil antenna 42a, and/or a far-field RF antenna 42b, as described earlier. ETS 40 may also include stimulation circuitry 44 able to form the stimulation pulses in accordance with a stimulation program, which circuitry may be similar to or comprise the same stimulation circuitry 28 present in the IPG 10. ETS 40 may also include a battery (not shown) for operational power.

FIG. 4 shows various external devices that can wirelessly communicate data with the IPG 10 and the ETS 40, including a patient, hand-held external controller 45, and a clinician programmer 50. Both of devices 45 and 50 can be used to send a stimulation program to the IPG 10 or ETS 40—that is, to program their stimulation circuitries 28 and 44 to produce pulses with a desired shape and timing described earlier. Both devices 45 and 50 may also be used to adjust one or more stimulation parameters of a stimulation program that the IPG 10 or ETS 40 is currently executing. Devices 45 and 50 may also receive information from the IPG 10 or ETS 40, such as various status information, etc.

External controller 45 can be as described in U.S. Patent Application Publication 2015/0080982 for example, and may comprise either a dedicated controller configured to work with the IPG 10. External controller 45 may also comprise a general purpose mobile electronics device such as a mobile phone which has been programmed with a Medical Device Application (MDA) allowing it to work as a wireless controller for the IPG 10 or ETS 40, as described in U.S. Patent Application Publication 2015/0231402. External controller 45 includes a user interface, including means for entering commands (e.g., buttons or icons) and a display 46. The external controller 45's user interface enables a patient to adjust stimulation parameters, although it may have limited functionality when compared to the more-powerful clinician programmer 50, described shortly.

The external controller 45 can have one or more antennas capable of communicating with the IPG 10 and ETS 40. For example, the external controller 45 can have a near-field magnetic-induction coil antenna 47a capable of wirelessly communicating with the coil antenna 26a or 42a in the IPG 10 or ETS 40. The external controller 45 can also have a far-field RF antenna 47b capable of wirelessly communicating with the RF antenna 26b or 42b in the IPG 10 or ETS 40.

The external controller 45 can also have control circuitry 48 such as a microprocessor, microcomputer, an FPGA, other digital logic structures, etc., which is capable of executing instructions an electronic device. Control circuitry 48 can for example receive patient adjustments to stimulation parameters, and create a stimulation program to be wirelessly transmitted to the IPG 10 or ETS 40.

Clinician programmer 50 is described further in U.S. Patent Application Publication 2015/0360038, and is only briefly explained here. The clinician programmer 50 can comprise a computing device 51, such as a desktop, laptop, or notebook computer, a tablet, a mobile smart phone, a Personal Data Assistant (PDA)-type mobile computing device, etc. In FIG. 4, computing device 51 is shown as a laptop computer that includes typical computer user interface means such as a screen 52, a mouse, a keyboard, speakers, a stylus, a printer, etc., not all of which are shown for convenience. Also shown in FIG. 4 are accessory devices for the clinician programmer 50 that are usually specific to its operation as a stimulation controller, such as a communication “wand” 54, and a joystick 58, which are coupleable to suitable ports on the computing device 51, such as USB ports 59 for example.

The antenna used in the clinician programmer 50 to communicate with the IPG 10 or ETS 40 can depend on the type of antennas included in those devices. If the patient's IPG 10 or ETS 40 includes a coil antenna 26a or 42a, wand 54 can likewise include a coil antenna 56a to establish near-filed magnetic-induction communications at small distances. In this instance, the wand 54 may be affixed in close proximity to the patient, such as by placing the wand 54 in a belt or holster wearable by the patient and proximate to the patient's IPG 10 or ETS 40.

If the IPG 10 or ETS 40 includes an RF antenna 26b or 42b, the wand 54, the computing device 51, or both, can likewise include an RF antenna 56b to establish communication with the IPG 10 or ETS 40 at larger distances. (Wand 54 may not be necessary in this circumstance). The clinician programmer 50 can also establish communication with other devices and networks, such as the Internet, either wirelessly or via a wired link provided at an Ethernet or network port.

To program stimulation programs or parameters for the IPG 10 or ETS 40, the clinician interfaces with a clinician programmer graphical user interface (GUI) 64 provided on the display 52 of the computing device 51. As one skilled in the art understands, the GUI 64 can be rendered by execution of clinician programmer software 66 on the computing device 51, which software may be stored in the device's non-volatile memory 68. One skilled in the art will additionally recognize that execution of the clinician programmer software 66 in the computing device 51 can be facilitated by control circuitry 70 such as a microprocessor, microcomputer, an FPGA, other digital logic structures, etc., which is capable of executing programs in a computing device. Such control circuitry 70, in addition to executing the clinician programmer software 66 and rendering the GUI 64, can also enable communications via antennas 56a or 56b to communicate stimulation parameters chosen through the GUI 64 to the patient's IPG 10.

A portion of the GUI 64 is shown in one example in FIG. 5. One skilled in the art will understand that the particulars of the GUI 64 will depend on where clinician programmer software 66 is in its execution, which will depend on the GUI selections the clinician has made. FIG. 5 shows the GUI 64 at a point allowing for the setting of stimulation parameters for the patient and for their storage as a stimulation program. To the left a program interface 72 is shown, which as explained further in the '038 Publication allows for naming, loading and saving of stimulation programs for the patient. Shown to the right is a stimulation parameters interface 82, in which specific stimulation parameters (A, D, F, E, P) can be defined for a stimulation program. Values for stimulation parameters relating to the shape of the waveform (A; in this example, current), pulse width (PW), and frequency (F) are shown in a waveform parameter interface 84, including buttons the clinician can use to increase or decrease these values.

Stimulation parameters relating to the electrodes 16 (the electrodes E activated and their polarities P), are made adjustable in an electrode parameter interface 86. Electrode stimulation parameters are also visible and can be manipulated in a leads interface 92 that displays the leads 15 (or 15′) in generally their proper position with respect to each other, for example, on the left and right sides of the spinal column. A cursor 94 (or other selection means such as a mouse pointer) can be used to select a particular electrode in the leads interface 92. Buttons in the electrode parameter interface 86 allow the selected electrode (including the case electrode, Ec) to be designated as an anode, a cathode, or off. The electrode parameter interface 86 further allows the relative strength of anodic or cathodic current of the selected electrode to be specified in terms of a percentage, X. This is particularly useful if more than one electrode is to act as an anode or cathode at a given time, as explained in the '038 Publication. In accordance with the example waveforms shown in FIG. 2, as shown in the leads interface 92, electrode E5 has been selected as the only anode to source current, and this electrode receives X=100% of the specified anodic current, +A. Likewise, electrode E4 has been selected as the only cathode to sink current, and this electrode receives X=100% of that cathodic current, −A.

The GUI 64 as shown specifies only a pulse width PW of the first pulse phase 30a. The clinician programmer software 66 that runs and receives input from the GUI 64 will nonetheless ensure that the IPG 10 and ETS 40 are programmed to render the stimulation program as biphasic pulses if biphasic pulses are to be used. For example, the clinician programming software 66 can automatically determine durations and amplitudes for both of the pulse phases 30a and 30b (e.g., each having a duration of PW, and with opposite polarities +A and −A). An advanced menu 88 can also be used (among other things) to define the relative durations and amplitudes of the pulse phases 30a and 30b, and to allow for other more advance modifications, such as setting of a duty cycle (on/off time) for the stimulation pulses, and a ramp-up time over which stimulation reaches its programmed amplitude (A), etc. A mode menu 90 allows the clinician to choose different modes for determining stimulation parameters. For example, as described in the '038 Publication, mode menu 90 can be used to enable electronic trolling, which comprises an automated programming mode that performs current steering along the electrode array by moving the cathode in a bipolar fashion.

While GUI 64 is shown as operating in the clinician programmer 50, the user interface of the external controller 45 may provide similar functionality.

### SUMMARY

Disclosed herein are methods for providing sub-perception electrical stimulation to a patient's spinal cord, the methods comprising: using one or more electrodes implanted within the patient's spinal column to provide a plurality of electrical pulses to the patient's spinal cord, wherein of each of the pulses are below the patient's perception threshold, wherein each pulse comprises an amplitude and a pulse width, and wherein the amplitudes of the plurality of pulses are ramped from a first amplitude value to second amplitude value at a rate of no more than 3 mA/s over a first duration. According to some embodiments, the first amplitude value is less than the second amplitude value. According to some embodiments, the second amplitude value is 80% or less than an amplitude value that causes paresthesia in the patient. According to some embodiments, the first amplitude value is greater than the second amplitude value. According to some embodiments, the method comprises providing no stimulation for a second duration. According to some embodiments, the second duration is at least one second. According to some embodiments, the pulse widths of the plurality of pulses vary over the first duration. According to some embodiments, the amplitudes of the plurality of pulses increase and the pulse widths of the plurality of pulses decrease over the first duration. According to some embodiments, the amplitudes of the plurality of pulses decrease and the pulse widths of the plurality of pulses increase over the first duration. According to some embodiments, each of the electrical pulses are biphasic pulses. According to some embodiments, the one or more of the electrodes comprise at least three anodes and at least three cathodes. According to some embodiments, the anodes are configured as a first three or more adjacent electrodes and the cathodes are each configured as a second set of three or more adjacent electrodes. According to some embodiments, the anodes each share an anodic current fractionalized among each of the first three or more adjacent electrodes, and the cathodes each share a cathodic current fractionalized among each of the second three or more adjacent electrodes. According to some embodiments, the anodic current is fractionalized equally among the first three or more adjacent electrodes and the cathodic current is fractionalized equally among the second three or more adjacent electrodes. According to some embodiments, the first set of adjacent electrodes comprises one or more rostral anodes, one or more middle anodes, and one or more caudal anodes, the rostral anodes and the caudal anodes each share more of the anodic current than the middle anodes, the second set of adjacent electrodes comprises one or more rostral cathodes, one or more middle cathodes and one or more caudal cathodes, and the rostral cathodes and the caudal cathodes each share more cathodic current than the middle cathodes.

Also disclosed herein is a system, comprising: a spinal cord stimulator comprising: stimulation circuitry programmed to generate a plurality of electrical stimulation pulses at a plurality of electrodes, wherein each of the pulses have a shape comprising an amplitude and a pulse width, wherein each of the pulses are configured to be below the patient's perception threshold, and wherein the amplitudes of the plurality of pulses are ramped from a first amplitude value to second amplitude value at a rate of no more than 3 mA/s. According to some embodiments, the first amplitude value is less than the second amplitude value. According to some embodiments, the second amplitude value is 80% or less than an amplitude value that causes paresthesia in the patient. According to some embodiments, the first amplitude value is greater than the second amplitude value. According to some embodiments, the pulse widths of the plurality of pulses vary over the first duration. According to some embodiments, the amplitudes of the plurality of pulses increase and the pulse widths of the plurality of pulses decrease over the first duration. According to some embodiments, the amplitudes of the plurality of pulses decrease and the pulse widths of the plurality of pulses increase over the first duration. According to some embodiments, each of the electrical pulses are biphasic pulses. According to some embodiments, the one or more of the electrodes comprise at least three anodes and at least three cathodes. According to some embodiments, the anodes are configured as a first three or more adjacent electrodes and the cathodes are each configured as a second set of three or more adjacent electrodes. According to some embodiments, the anodes each share an anodic current fractionalized among each of the first three or more adjacent electrodes, and the cathodes each share a cathodic current fractionalized among each of the second three or more adjacent electrodes. According to some embodiments, the anodic current is fractionalized equally among the first three or more adjacent electrodes and the cathodic current is fractionalized equally among the second three or more adjacent electrodes. According to some embodiments, the first set of adjacent electrodes comprises one or more rostral anodes, one or more middle anodes, and one or more caudal anodes, the rostral anodes and the caudal anodes each share more of the anodic current than the middle anodes, the second set of adjacent electrodes comprises one or more rostral cathodes, one or more middle cathodes and one or more caudal cathodes, and the rostral cathodes and the caudal cathodes each share more cathodic current than the middle cathodes.

Also disclosed herein is a method for providing sub-perception electrical stimulation to a patient's spinal cord, the method comprising: using a plurality of electrodes implanted within the patient's spinal column to provide a plurality of electrical pulses to the patient's spinal cord for a first duration, wherein each electrical pulse is below the patient's perception threshold, wherein each pulse comprises an amplitude and a pulse width, wherein one or more of the amplitude and/or pulse width varies over the first duration, and wherein the plurality of electrodes comprises: at least three anodes configured as a first set of three or more adjacent electrodes, each sharing an anodic current and comprising one or more rostral anodes, one or more middle anodes, and one or more caudal anodes, wherein the rostral anodes and the caudal anodes each share more of the anodic current than the middle anodes, at least three cathodes configured as a second set of three or more adjacent electrodes, each sharing a cathodic current, wherein the second set of three or more adjacent electrodes comprises one or more rostral cathodes, one or more middle cathodes, and one or more caudal cathodes, wherein the rostral cathodes and the caudal cathodes each share more of the cathodic current than the middle cathodes. According to some embodiments, the amplitudes of the plurality of pulses ramp from a first amplitude value to a second amplitude value over the first duration. According to some embodiments, the first amplitude value is less than the second amplitude value. According to some embodiments, the second amplitude value is 80% or less than an amplitude value that causes paresthesia in the patient. According to some embodiments, the first amplitude value is greater than the second amplitude value. According to some embodiments, the pulse widths of the plurality of pulses vary over the first duration. According to some embodiments, the amplitudes of the plurality of pulses increase and the pulse widths of the plurality of pulses decrease over the first duration. According to some embodiments, the amplitudes of the plurality of pulses decrease and the pulse widths of the plurality of pulses increase over the first duration. According to some embodiments, each of the electrical pulses are biphasic pulses.

Also disclosed herein is a system, comprising: a spinal cord stimulator comprising: stimulation circuitry programmed to generate a plurality of electrical stimulation pulses at a plurality of electrodes, wherein each of the pulses have a shape comprising an amplitude and a pulse width, wherein each of the pulses are configured to be below the patient's perception threshold, and wherein one or more of the amplitude and/or pulse width varies over a first duration, and wherein the plurality of electrodes comprises: at least three anodes configured as a first set of three or more adjacent electrodes, each sharing an anodic current and comprising one or more rostral anodes, one or more middle anodes, and one or more caudal anodes, wherein the rostral anodes and the caudal anodes each share more of the anodic current than the middle anodes, at least three cathodes configured as a second set of three or more adjacent electrodes, each sharing a cathodic current, wherein the second set of three or more adjacent electrodes comprises one or more rostral cathodes, one or more middle cathodes, and one or more caudal cathodes, wherein the rostral cathodes and the caudal cathodes each share more of the cathodic current than the middle cathodes. According to some embodiments, the amplitudes of the plurality of pulses ramp from a first amplitude value to a second amplitude value over the first duration. According to some embodiments, the first amplitude value is less than the second amplitude value. According to some embodiments, the second amplitude value is 80% or less than an amplitude value that causes paresthesia in the patient. According to some embodiments, the first amplitude value is greater than the second amplitude value. According to some embodiments, the pulse widths of the plurality of pulses vary over the first duration. According to some embodiments, the amplitudes of the plurality of pulses increase and the pulse widths of the plurality of pulses decrease over the first duration. According to some embodiments, the amplitudes of the plurality of pulses decrease and the pulse widths of the plurality of pulses increase over the first duration. According to some embodiments, each of the electrical pulses are biphasic pulses.

Also disclosed herein is a method for providing sub-perception electrical stimulation to a patient's spinal cord, the method comprising: using a plurality of electrodes implanted within the patient's spinal column to provide a plurality of electrical pulses to the patient's spinal cord for a first duration, wherein each pulse comprises an amplitude and a pulse width, wherein the amplitudes vary from a first amplitude value to a second amplitude value over the duration, wherein the pulse widths vary from a first pulse width value to a second pulse width value over the duration, and wherein the first duration is at least one second. According to some embodiments, the amplitudes of the plurality of pulses increase and the pulse widths of the plurality of pulses decrease over the duration. According to some embodiments, the amplitudes of the plurality of pulses decrease and the pulse widths of the plurality of pulses increase over the duration. According to some embodiments, each of the pulses is below the patient's perception threshold. According to some embodiments, the highest of the first amplitude value or the second amplitude value is 80% or less than an amplitude that causes paresthesia in the patient. According to some embodiments, each of the electrical pulses are biphasic pulses. According to some embodiments, the one or more of the electrodes comprise at least three anodes and at least three cathodes. According to some embodiments, the anodes are configured as a first three or more adjacent electrodes and the cathodes are each configured as a second set of three or more adjacent electrodes. According to some embodiments, the anodes each share an anodic current fractionalized among each of the first three or more adjacent electrodes, and the cathodes each share a cathodic current fractionalized among each of the second three or more adjacent electrodes. According to some embodiments, the anodic current is fractionalized equally among the first three or more adjacent electrodes and the cathodic current is fractionalized equally among the second three or more adjacent electrodes. According to some embodiments, the first set of adjacent electrodes comprises one or more rostral anodes, one or more middle anodes, and one or more caudal anodes, the rostral anodes and the caudal anodes each share more of the anodic current than the middle anodes, the second set of adjacent electrodes comprises one or more rostral cathodes, one or more middle cathodes and one or more caudal cathodes, and the rostral cathodes and the caudal cathodes each share more cathodic current than the middle cathodes.

Also disclosed herein is a system, comprising: a spinal cord stimulator comprising: stimulation circuitry programmed to generate a plurality of electrical stimulation pulses at a plurality of electrodes, wherein each of the pulses have a shape comprising an amplitude and a pulse width, wherein the amplitudes vary from a first amplitude value to a second amplitude value over a duration, wherein the pulse widths vary from a first pulse width value to a second pulse width value over the duration, and wherein the first duration is at least one second. According to some embodiments, the amplitudes of the plurality of pulses increase and the pulse widths of the plurality of pulses decrease over the duration. According to some embodiments,

the amplitudes of the plurality of pulses decrease and the pulse widths of the plurality of pulses increase over the duration. According to some embodiments, each of the pulses is below the patient's perception threshold. According to some embodiments, the highest of the first amplitude value or the second amplitude value is 80% or less than an amplitude that causes paresthesia in the patient. According to some embodiments, each of the electrical pulses are biphasic pulses. According to some embodiments, the one or more of the electrodes comprise at least three anodes and at least three cathodes. According to some embodiments, the anodes are configured as a first three or more adjacent electrodes and the cathodes are each configured as a second set of three or more adjacent electrodes. According to some embodiments, the anodes each share an anodic current fractionalized among each of the first three or more adjacent electrodes, and the cathodes each share a cathodic current fractionalized among each of the second three or more adjacent electrodes. According to some embodiments, the anodic current is fractionalized equally among the first three or more adjacent electrodes and the cathodic current is fractionalized equally among the second three or more adjacent electrodes. According to some embodiments, the first set of adjacent electrodes comprises one or more rostral anodes, one or more middle anodes, and one or more caudal anodes, the rostral anodes and the caudal anodes each share more of the anodic current than the middle anodes, the second set of adjacent electrodes comprises one or more rostral cathodes, one or more middle cathodes and one or more caudal cathodes, and the rostral cathodes and the caudal cathodes each share more cathodic current than the middle cathodes.

The invention may also reside in the form of a programed external device (via its control circuitry) for carrying out the above methods, a programmed IPG or ETS (via its control circuitry) for carrying out the above methods, a system including a programmed external device and IPG or ETS for carrying out the above methods, or as a computer readable media for carrying out the above methods stored in an external device or IPG or ETS.

### BRIEF DESCRIPTION OF THE DRAWINGS

FIG. 1 shows an Implantable Pulse Generator (IPG) useable for Spinal Cord Stimulation (SC S), in accordance with the prior art.

FIG. 2 shows an example of stimulation pulses producible by the IPG, in accordance with the prior art.

FIG. 3 shows use of an External Trial Stimulator (ETS) useable to provide stimulation before implantation of an IPG, in accordance with the prior art.

FIG. 4 shows various external devices capable of communicating with and programming stimulation in an IPG and ETS, in accordance with the prior art.

FIG. 5 shows a Graphical User Interface (GUI) of a clinician programmer external device for setting or adjusting stimulation parameters, in accordance with the prior art.

FIGS. 6A and 6B show sweet spot searching to determine effective electrodes for a patient using a movable sub-perception bipole.

FIG. 7 shows a virtual bipole for treating an area of neural tissue believed to contain the center of a patient's pain.

FIG. 8 shows stimulation circuitry useable in the IPG or ETS capable of providing Multiple Independent Current Control to independently set the current at each of the electrodes.

FIG. 9 shows a virtual bipole for treating an area of neural tissue believed to contain the center of a patient's pain.

FIGS. 10A and 10B show examples of sub-perception stimulation waveforms wherein the amplitudes ramp between a first value and a second value.

FIG. 11 shows an example of a stimulation waveform wherein both the amplitude and the pulse width varies.

### DETAILED DESCRIPTION

While Spinal Cord Stimulation (SCS) therapy can be an effective means of alleviating a patient's pain, such stimulation can also cause paresthesia. Paresthesia is a sensation such as tingling, prickling, heat, cold, etc. that can accompany SCS therapy. Generally, the effects of paresthesia are mild, or at least are not overly concerning to a patient. Moreover, paresthesia is generally a reasonable tradeoff for a patient whose chronic pain has now been brought under control by SCS therapy. Some patients even find paresthesia comfortable and soothing.

Nonetheless, at least for some patients, SCS therapy would ideally provide complete pain relief without paresthesia—what is often referred to as “sub-perception” or sub-threshold therapy that a patient cannot feel. Effective sub-perception therapy may provide pain relief without paresthesia by issuing stimulation pulses at higher frequencies. Unfortunately, such higher-frequency stimulation may require more power, which tends to drain the battery 14 of the IPG 10. See, e.g., U.S. Patent Application Publication 2016/0367822. If an IPG's battery 14 is a primary cell and not rechargeable, high-frequency stimulation means that the IPG 10 will need to be replaced more quickly. Alternatively, if an IPG battery 14 is rechargeable, the IPG 10 will need to be charged more frequently, or for longer periods of time. Either way, the patient is inconvenienced.

In an SCS application, it is desirable to determine a stimulation program that will be effective for each patient. A significant part of determining an effective stimulation program is to determine a “sweet spot” for stimulation in each patient, i.e., to select which electrodes should be active (E) and with what polarities (P) and relative amplitudes (X %) to recruit and thus treat a neural site at which pain originates in a patient. Selecting electrodes proximate to this neural site of pain can be difficult to determine, and experimentation is typically undertaken to select the best combination of electrodes to provide a patient's therapy.

As described in U.S. patent application Ser. No. 16/419,879, filed May 22, 2019, which is hereby expressly incorporated by reference, selecting electrodes for a given patient can be even more difficult when sub-perception therapy is used, because the patient does not feel the stimulation, and therefore it can be difficult for the patient to feel whether the stimulation is “covering” his pain and therefore whether selected electrodes are effective. Further, sub-perception stimulation therapy may require a “wash in” period before it can become effective. A wash in period can take up to a day or more, and therefore sub-perception stimulation may not be immediately effective, making electrode selection more difficult.

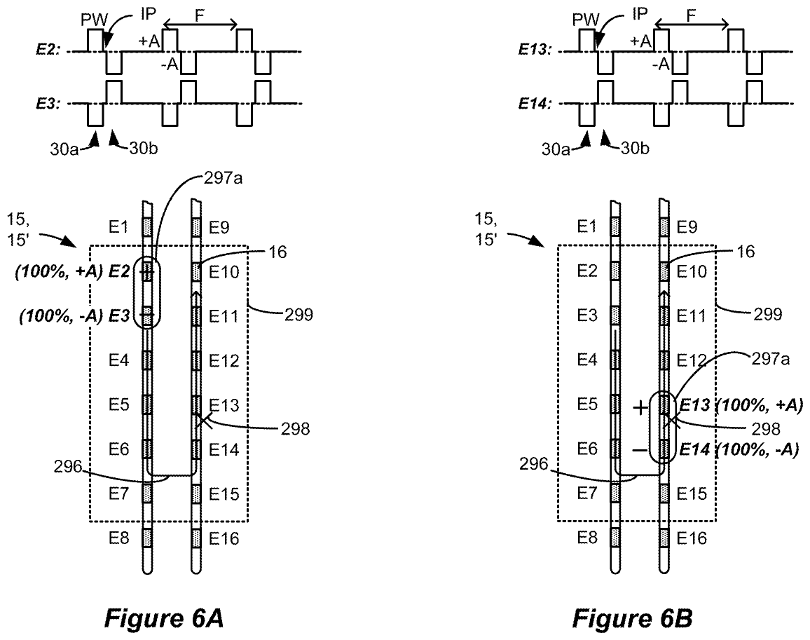

FIGS. 6A and 6B briefly explain the '879 application's technique for a sweet spot search, i.e., how electrodes can be selected that are proximate to a site of neural pain 298 in a patient, when sub-perception stimulation is used. The technique of FIGS. 6A and 6B is particularly useful in a trial setting after a patient is first implanted with an electrode array 15 (or 15′), i.e., after receiving their IPG or ETS.

In the example shown, it is assumed that a pain site 298 is likely within a tissue region 299. Such region 299 may be deduced by a clinician based on the patient symptoms, e.g., by understanding which electrodes are proximate to certain vertebrae (not shown), such as within the T7-T10 interspace, as just one example. Of course, the region 299 may be any portion of the spine. In the example shown, region 299 is bounded by electrodes E2, E7, E15, and E10, meaning that electrodes outside of this region (e.g., E1, E8, E9, E16) are unlikely to have an effect on the patient's symptoms. Therefore, these electrodes may not be selected during the sweet spot search depicted in FIG. 6A, as explained further below.

In FIG. 6A, a sub-perception bipole 297a is selected, in which one electrode (e.g., E2) is selected as an anode that will source a positive current (+A) to the patient's tissue, while another electrode (e.g., E3) is selected as a cathode that will sink a negative current (−A) from the tissue. This is similar to what was illustrated earlier with respect to FIG. 2, and biphasic stimulation pulses can be used employing active charge recovery. Because the bipole 297a provides sub-perception stimulation, the amplitude A used during the sweet spot search is titrated down until the patient no longer feels paresthesia. This sub-perception bipole 297a is provided to the patient for a duration, such as a few days, which allows the sub-perception bipole's potential effectiveness to “wash in,” and allows the patient to provide feedback concerning how well the bipole 297a is helping their symptoms. Such patient feedback can comprise a pain scale ranking. For example, the patient can rank their pain on a scale from 1-10 using a Numerical Rating Scale (NRS) or the Visual Analogue Scale (VAS), with 1 denoting no or little pain and 10 denoting a worst pain imaginable. As discussed in the '539 application, such pain scale ranking can be entered into the patient's external controller 45.

After the bipole 297a is tested at this first location, a different combination of electrodes is chosen (anode electrode E3, cathode electrode E4), which moves the location of the bipole 297 in the patient's tissue. Again, the amplitude of the current A may need to be titrated to an appropriate sub-perception level. In the example shown, the bipole 297a is moved down one electrode lead, and up the other, as shown by path 296 in the hope of finding a combination of electrodes that covers the pain site 298. In the example of FIGS. 6A and 6B, given the pain site 298's proximity to electrodes E13 and E14, it might be expected that a bipole 297a at those electrodes, as reflected in FIG. 6B, will provide the best relief for the patient, as reflected by the patient's pain score rankings. The particular stimulation parameters chosen when forming bipole 297a can be selected at the GUI 64 of the clinician programmer 50 or other external device (such as a patient external controller 45) and wirelessly telemetered to the patient's IPG or ETS for execution. Note, as used herein, the term spinal cord stimulator may refer to both an IPG or an ETS.

While the sweet spot search of FIGS. 6A and 6B can be effective, it can also take a significantly long time when sub-perception stimulation is used. As noted, sub-perception stimulation is provided at each bipole 297 location for a number of days, and because a large number of bipole locations are chosen, the entire sweep spot search can take up to a month to complete.

The inventor has discovered that effective sub-perception stimulation can be achieved without conducting the cumbersome sweet spot search described with respect to FIGS. 6A and 6B. An aspect of this disclosure is directed to sub-perception stimulation using virtual poles that encompass a plurality of electrodes and that are large enough to cover the patient's pain site without extensive sweet spot searching. Virtual poles are discussed further in U.S. Pat. No. 10,881,859, issued Jan. 5, 2021, which is incorporated herein by reference in its entirety, and thus virtual poles are only briefly explained here. Forming virtual poles is assisted if the stimulation circuitry 28 or 44 used in the IPG or ETS is capable of independently setting the current at any of the electrodes—what is sometimes known as a Multiple Independent Current Control (MICC), which is explained further below with reference to FIG. 8.

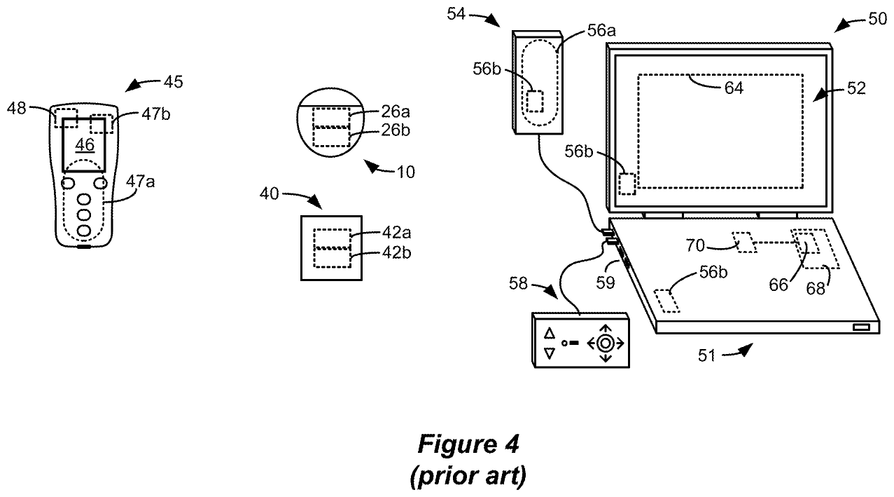

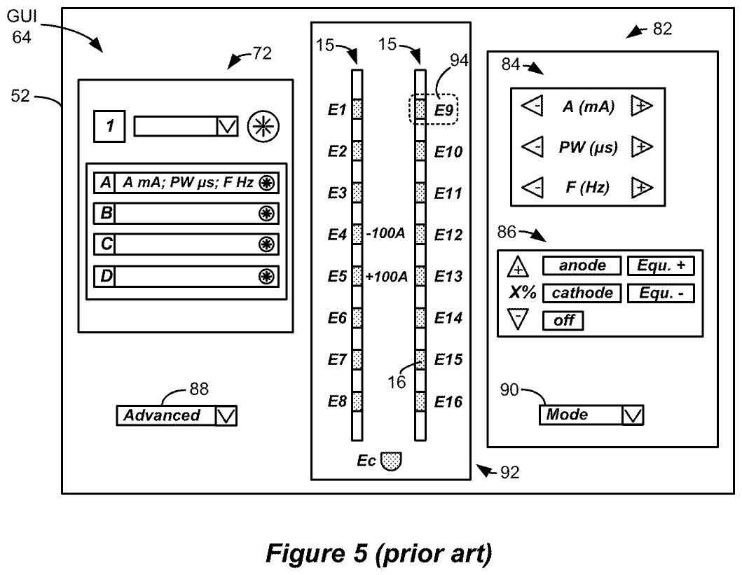

When a virtual bipole is used, the GUI 64 (FIG. 5) of the clinician programmer 50 (FIG. 4) can be used to define an anode pole (+) and a cathode pole (−) at positions that may not necessarily correspond to the position of the physical electrodes. The control circuitry 70 in the clinician programmer 50 can compute from these positions and from other tissue modeling information which physical electrodes will need to be selected and with what amplitudes to form the virtual anode and virtual cathode at the designated positions. As described earlier, amplitudes at selected electrodes may be expressed as a percentage X % of the total current amplitude A specified at the GUI 64 of the clinician programmer 50.

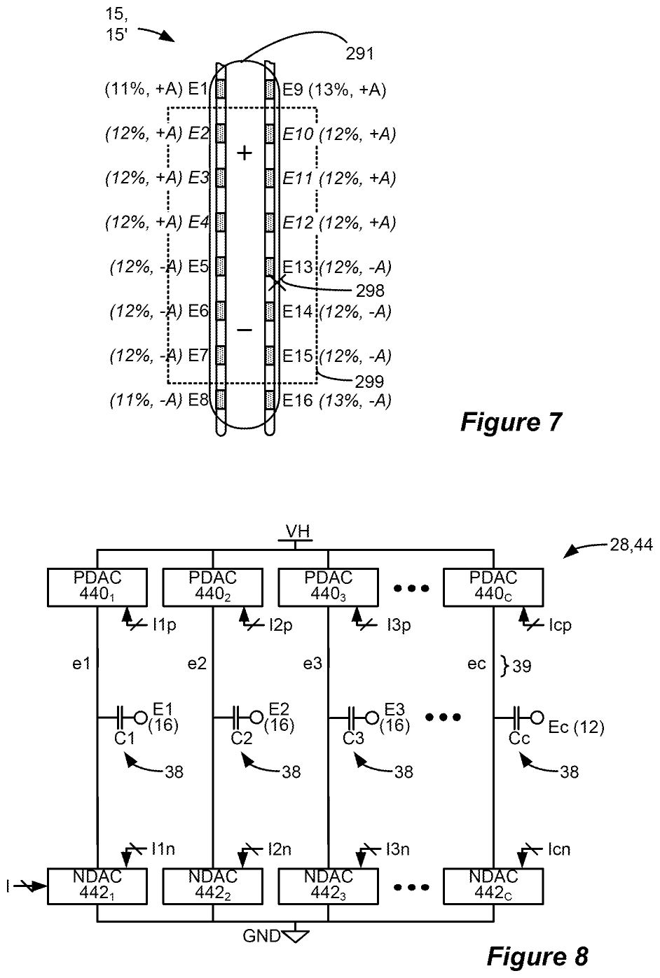

According to some embodiments, the clinician programmer 50 can be used to assign percentages of the anodic and cathodic currents to be shared by the various electrodes. For example, the user may use the GUI 64 to select particular electrodes and assign the percentages of the anodic or cathodic currents that those electrodes should share. The inventor has discovered that effective sub-perception stimulation can be achieved when current is shared among a relatively large number of electrodes so as to generate a virtual pole that covers the region of the patient's pain. FIG. 7 illustrates one example of this approach. Assume that the patient's pain site 298 is within a tissue region 299, as was described above with respect to FIGS. 6A and 6B. The exact pain site 298 is not known. As described above, a cumbersome sweet spot searching routine could be performed to identify a specific, focused bipole that covers that pain site. However, the inventor has discovered that using a plurality of anodes to share anodic current and a plurality of cathodes to share cathodic current in or near the tissue region 299 provides a virtual bipole 291 that covers the pain site 298 without having to use a sweet spot search. In FIG. 7, current is shared among each of electrodes E1-E16 near and within the tissue region 299 to make the virtual bipole 291. Specifically, anodic current is shared essentially equally among electrodes E1-E4 and E9-E12 to provide a virtual anode pole, and cathodic current is shared essentially equally among electrodes E5-E8 and E13-E16 to provide a virtual cathode pole. The virtual anode and cathode poles combine to provide the virtual bipole 291. Note that if biphasic pulses are used, then the polarities of the electrodes switch during the second phase of the pulse. Other electrode configurations for providing a large virtual bipole are possible and some other configurations will be described below. According to some embodiments, anodic current is shared or fractionalized among three or more electrodes to make a virtual anode and cathodic current is shared of fractionalized among three or more electrodes to make a virtual cathode. According to some embodiments, the three or more anodes may be adjacent with each other and the three or more cathodes may be adjacent with each other. According to some embodiments, the total current amplitude shared amongst the electrodes is below the perception threshold of the patient.

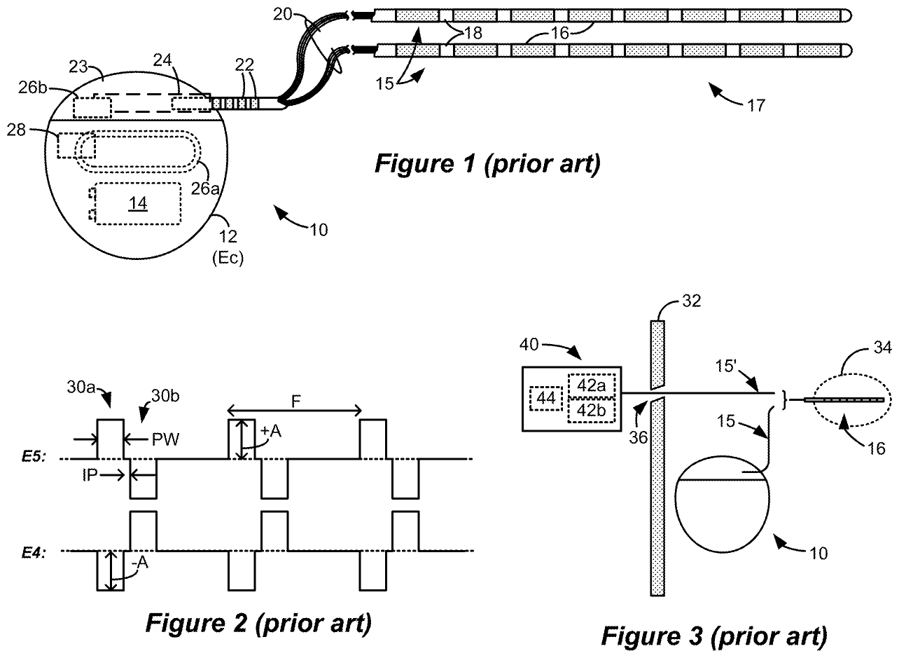

As mentioned above, stimulation circuitry capable of Multiple Independent Current Control (MICC) can be used to make the virtual bipoles, as described herein. Multiple Independent Current Control (MICC) is explained in one example with reference to FIG. 8, which shows the stimulation circuitry 28 (FIG. 1) or 44 (FIG. 3) in the IPG or ETS used to form prescribed stimulation at a patient's tissue. The stimulation circuitry 28 or 44 can control the current or charge at each electrode independently, and using GUI 64 (FIG. 5) allows the current or charge to be steered to different electrodes, which is useful for example when fractionalizing the total anodic and cathodic current among the electrodes, as shown in FIG. 7. The stimulation circuitry 28 or 44 includes one or more current sources 440ᵢ and one or more current sinks 442ᵢ. The sources and sinks 440ᵢ and 442ᵢ can comprise Digital-to-Analog converters (DACs), and may be referred to as PDACs 440ᵢ and NDACs 442ᵢ in accordance with the Positive (sourced, anodic) and Negative (sunk, cathodic) currents they respectively issue. In the example shown, a NDAC/PDAC 440ᵢ/442ᵢ pair is dedicated (hardwired) to a particular electrode node ei 39. Each electrode node ei 39 is preferably connected to an electrode Ei 16 via a DC-blocking capacitor Ci 38, which act as a safety measure to prevent DC current injection into the patient, as could occur for example if there is a circuit fault in the stimulation circuitry 28 or 44. PDACs 440ᵢ and NDACs 442ᵢ can also comprise voltage sources.

Proper control of the PDACs 440ᵢ and NDACs 442ᵢ via GUI 64 allows any of the electrodes 16 and the case electrode Ec 12 to act as anodes or cathodes to create a current through a patient's tissue. Such control preferably comes in the form of digital signals Tip and Iin that set the anodic and cathodic current at each electrode Ei. If for example it is desired to set electrode E1 as an anode with a current of +3 mA, and to set electrodes E2 and E3 as cathodes with a current of −1.5 mA each, control signal I1p would be set to the digital equivalent of 3 mA to cause PDAC 440₁ to produce +3 mA, and control signals I2n and I3n would be set to the digital equivalent of 1.5 mA to cause NDACs 442₂ and 442₃ to each produce −1.5 mA. Note that definition of these control signals can also occur using the programmed amplitude A and percentage X % set in the GUI 64. For example, A may be set to 3 mA, with E1 designated as an anode with X=100%, and with E2 and E3 designated at cathodes with X=50%. Alternatively, the control signals may not be set with a percentage, and instead the GUI 64 can simply prescribe the current that will appear at each electrode at any point in time.

In short, the GUI 64 may be used to independently set the current at each electrode, or to steer the current between different electrodes. This is particularly useful in forming virtual bipoles, which as explained earlier involve activation of more than two electrodes. MICC also allows more sophisticated electric fields to be formed in the patient's tissue.

Other stimulation circuitries 28 can also be used to implement MICC. In an example not shown, a switching matrix can intervene between the one or more PDACs 440ᵢ and the electrode nodes ei 39, and between the one or more NDACs 442ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, 10,912,942, U.S. Patent Application Publication 2018/0071513, and 2018/0071520.

Much of the stimulation circuitry 28 or 44, including the PDACs 440ᵢ and NDACs 442ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with the IPG's or ETS's telemetry antennas), circuitry for generating the compliance voltage VH that powers the stimulation circuitry, various measurement circuits, etc.

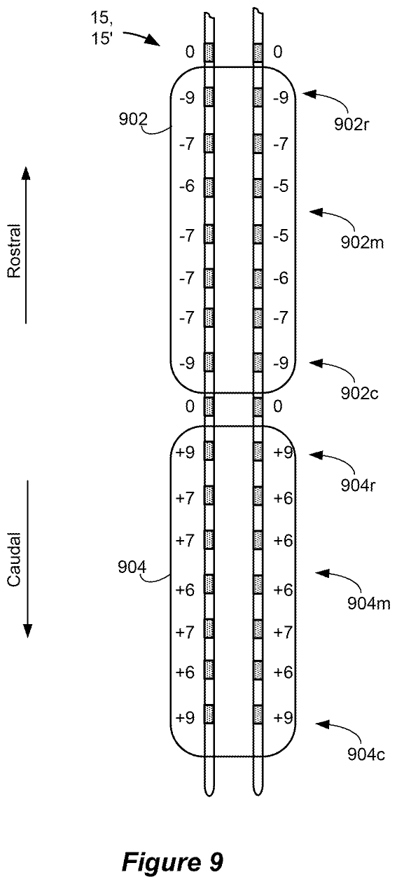

FIG. 9 illustrates another electrode configuration for providing a large virtual dipole for providing sub-perception stimulation therapy. The electrode leads 15 (or 15′) each comprise 16 electrodes in this illustration. For the sake of clarity, the electrodes are not individually labeled (e.g., by E1, E2, etc.). The numbers by each electrode denote the percentage of current each electrodes share. In the illustration, cathodic current is shared among a plurality of cathodes to provide a virtual cathode pole 902. According to some embodiments, the cathodes may comprise three or more electrodes that may be adjacent to each other. Anodic current is shared among a plurality of anodes to provide a virtual anode pole 904. According to some embodiments, the anodes may comprise three or more electrodes, which may be adjacent to each other. The virtual cathode pole and the virtual anode pole combine to form a virtual bipole that essentially covers the entire length of the leads in the illustrated example.

Notice in FIG. 9 that the virtual cathode pole 902 comprises rostral cathodes 902r, middle cathodes 902m, and caudal cathodes 902c. Likewise, the virtual anode pole 904 comprises rostral anodes 904r, middle anodes 904m, and caudal anodes 904c. In the illustrated embodiment, the rostral cathodes 902r and caudal cathodes 902c each share more cathodic current than do the middle cathodes 902m. Similarly, the rostral and caudal anodes (904r and 904c, respectively) share more of the anodic current than do the middle anodes 904m. Stated differently, the distribution of the current among the electrodes of each of the virtual poles is weighted m or heavily among the electrodes at the edges of the poles, compared to the electrodes in the middle of the poles. The inventor has found that such current distributions may provide more uniform electric fields in the patient's tissue.

As described above, using large virtual bipoles that cover a large region where the patient's pain site is expected to be located may obviate the need to do extensive tedious sweet spot searching. This can significantly accelerate the fitting process for the clinician and the patient. Another aspect of the fitting process is determining appropriate stimulation parameters to treat the patient's pain and to avoid unwanted side effects. Such stimulation parameters include parameters such as stimulation amplitude, pulse width, and the like. A traditional fitting process typically involves trying many different stimulation parameters to find the best stimulation amplitude, pulse width, etc. for the patient. That can be difficult and time consuming, especially with sub-perception therapy, because of the lack of paresthesia and delayed patient feedback due to the lengthy wash-in period discussed above.

The traditional fitting procedure assumes that there is one optimal sweet spot and set of stimulation parameters (amplitude, pulse width, etc.) for treating the patient. The techniques described above obviate the need for finding the optimal sweet spot by using a large virtual bipole that effectively covers a large area in the region of the patient's pain center. The inventor has also found that the need to find the perfect amplitude, pulse width, etc., may be obviated in some instances by ramping the neural dosage of stimulation provided to a patient over a significantly long duration.

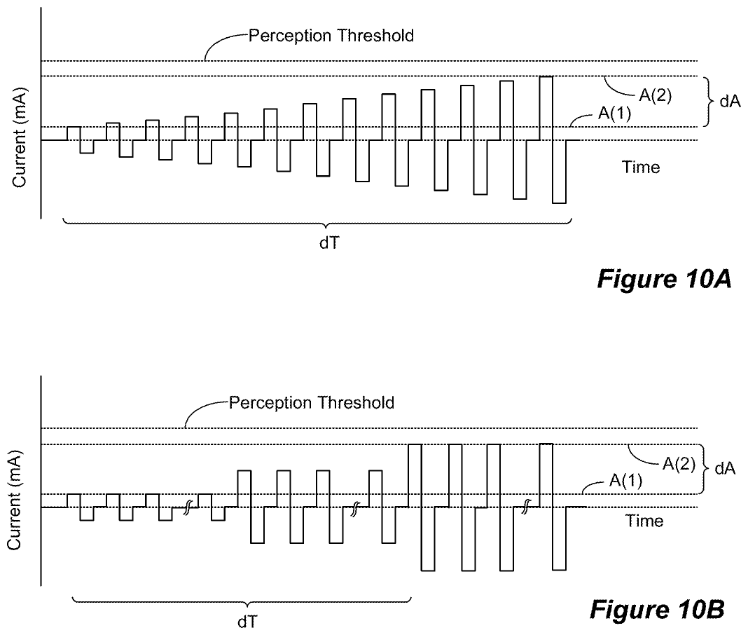

FIGS. 10A and 10B illustrate two embodiments of stimulation waveforms wherein the amplitude is ramped between a first amplitude A(1) and a second amplitude A(2) over a time duration dT. Note that only one “pole” of the stimulation is illustrated, which in this case is the anodic-first pole. A complementary cathodic-first waveform would typically be issued at one or more other electrodes to sink the current injected into the tissue by the illustrated waveform, as described above. Both A(1) and A(2) are below the patient's perception threshold. The clinician may determine the minimum stimulation amplitude at which the patient perceives the stimulation. The highest amplitude of the illustrated sub-perception waveform (A(2) in the illustration) may be set at about 80% of the patient's determined perception threshold amplitude value, for example.

Assume that the optimal stimulation amplitude is at some amplitude value between A(1) and A(2), but the actual value of the optimal amplitude is unknown. Rather than trying to identify a single optimum stimulation amplitude, ramping the amplitude slowly between A(1) and A(2) over a duration dT may provide effective pain-relieving stimulation. In FIG. 10A, the amplitude is ramped by increasing the amplitude with each pulse. In FIG. 10B, the amplitude is ramped by issuing a first plurality of pulses having a first amplitude, then a second plurality of pulses having a slightly higher amplitude, and repeating the process until the final amplitude A(2) is reached. In the embodiments illustrated in FIGS. 10A and 10B, the amplitudes are ramped from a lower amplitude value to a higher amplitude value (i.e., A(2)>A(1)). That is, the ramping rate dA/dT is positive. But other embodiments may involve ramping from a higher amplitude to a lower amplitude (i.e., A(2)<A(1)). In either case, the ramping function is typically a monotonic function.

The ramping of stimulation amplitude is described in the prior art. But typically, prior art modalities that involve the ramping of stimulation amplitude involve ramping the amplitude from an initial value to a final value over a duration that is on the order of milliseconds. By contrast, the paradigms described in this disclosure typically involve ramping the amplitude over a duration (dT) of a few seconds to tens of seconds. For example, the time duration dT may be about 4 seconds to about 20 seconds. Of course, the duration could be longer or shorter. Stated differently, the ramping rates (dA/dT) used in the methods described herein are typically much lower than those described in the prior art. Typically, IPGs such as those described herein, are capable of producing stimulation pulses on the order of about 12 mA. So, if the stimulation amplitude is ramped over the entire 12 mA range of the IPG over a duration of 4 seconds, then the ramp rate dA/dT is 3 mA/second. According to most embodiments described herein, the stimulation amplitude remains below the patient's perception threshold, which is likely well below 12 mA. Accordingly, the value of dA is typically well below the entire range provided by the IPG. For example, dA may more typically be about 6 mA or less. Likewise, the duration dT is typically longer, for example 3 seconds or longer. Ramping over a dT of 6 mA over a duration of 3 seconds yields a ramping rate (dA/dT) of 2 mA/second. Accordingly, embodiments of the disclosed methods involve ramping the amplitudes at a rate of less than 6 mA/second, or less than 4 mA/second, or less than 2 mA/second, or less than 1 mA/second. Once the ramping reaches the second (i.e., final) amplitude A(2), one more pulses of stimulation at the second amplitude may be delivered, followed by a duration of delivering no stimulation. The duration of delivering no stimulation may be on the order of one second, for example. The ramping sequence may begin again, using stimulation having the initial amplitude A(1). Alternatively, once the ramping reaches the final amplitude, the pattern may restart at the initial amplitude immediately without a period of delivering no stimulation. Still alternatively, once the ramping reaches the final amplitude, the amplitudes may ramp back down to the initial amplitude.

It should be noted that the currents of the waveforms illustrated in FIGS. 10A and 10B may be shared amongst multiple electrodes. For example, the currents can be shared among three or more electrodes to form large virtual poles to cover an area of the patient's tissue, as described above. It should also be noted that the waveforms illustrated in FIGS. 10A and 10B comprises symmetric biphasic pulses. However, different pulse shapes may be used. For example, monophasic pulses may be used, in which case, passive charge recovery may be used to recover charge from the patient's tissue. Asymmetric biphasic pulses may be used, for example, wherein the anodic and cathodic phases have different amplitudes and durations. The interval between the anodic and cathodic phases, i.e., the interphase interval (IPI), may be varied, as is known in the art.

Other parameters besides (or in addition to) the stimulation amplitude may be varied while delivering the stimulation. For example, the pulse width may be varied. It is believed that electrical stimulation generally recruits larger neural fibers more easily (or more quickly) than smaller fibers. Accordingly, stimulation having a relatively short pulse width is likely to recruit a higher percentage of larger fibers, whereas stimulation having a longer pulse width is likely to recruit more smaller fibers in addition to the larger fibers. Using traditional fitting paradigms, a clinician would endeavor to optimize the pulse width of the stimulation using a time-intensive trial-and-error process. The inventor has discovered that varying the stimulation pulse width while providing stimulation can be effective for providing the patient with beneficial therapy.

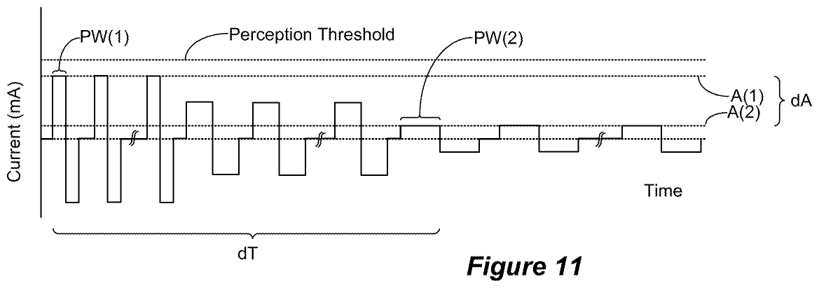

FIG. 11 illustrates a stimulation waveform wherein both the stimulation amplitude and the pulse width are varied. The amplitude of the pulses is varied (specifically, ramped) from an initial amplitude of A(1) to a final amplitude of A(2) over the duration dT. Note that in FIG. 11A(1) is greater than A(2). In other words, FIG. 11 illustrates an embodiment of ramping the amplitude using a negative rate (dA/dT), in contrast to the positive ramping rate illustrated in FIGS. 10A and 10B. The pulse width of the stimulation is varied from an initial pulse width of PW(1) to a final pulse width of PW(2). The pulse widths of the pulses are increased as the amplitudes are decreased. According to some embodiments, the amplitudes and pulse widths may each be varied so that the patient receives a constant dosage of current for each amplitude-pulse width combination. One example of a waveform wherein both the amplitude and the pulse widths of the pulses vary, is a waveform comprising a first plurality of pulses having amplitude of 6 mA and pulse widths of 100 μs, a second plurality of pulses having amplitudes of 3 mA and pulse widths of 200 μs, and a third plurality of pulses having amplitudes of 2 mA and pulse widths of 400 μs. Once the final plurality of pulses has been delivered, stimulation may be turned of for a duration and the sequence may be repeated. As discussed above, the currents of the illustrated waveforms may be shared among a plurality of anodes/cathodes to generate large virtual poles.

Various aspects of the disclosed techniques, including processes implementable in the IPG or ETS, or in external devices such as the clinician programmer or external controller to render and operate the GUI 64, can be formulated and stored as instructions in a computer-readable media associated with such devices, such as in a magnetic, optical, or solid state memory. The computer-readable media with such stored instructions may also comprise a device readable by the clinician programmer or external controller, such as in a memory stick or a removable disk, and may reside elsewhere. For example, the computer-readable media may be associated with a server or any other computer device, thus allowing instructions to be downloaded to the clinician programmer system or external controller or to the IPG or ETS, via the Internet for example.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for providing sub-perception electrical stimulation to a patient's spinal cord, the method comprising:
using one or more electrodes implanted within the patient's spinal column to provide a plurality of electrical pulses to the patient's spinal cord, wherein of each of the pulses are below the patient's perception threshold,
wherein each pulse comprises an amplitude and a pulse width, and
wherein the amplitudes of the plurality of pulses are ramped from a first amplitude value to second amplitude value at a rate of no more than 3 mA/s over a first duration.

2. The method of claim 1, wherein the first amplitude value is less than the second amplitude value.

3. The method of claim 2, wherein the second amplitude value is 80% or less than an amplitude value that causes paresthesia in the patient.

4. The method of claim 1, wherein the first amplitude value is greater than the second amplitude value.

5. The method of claim 1, further comprising providing no stimulation for a second duration and then repeating the steps of claim 1.

6. The method of claim 5, wherein the second duration is at least one second.

7. The method of claim 1, wherein the pulse widths of the plurality of pulses vary over the first duration.

8. The method of claim 7, wherein the amplitudes of the plurality of pulses increase and the pulse widths of the plurality of pulses decrease over the first duration.

9. The method of claim 7, wherein the amplitudes of the plurality of pulses decrease and the pulse widths of the plurality of pulses increase over the first duration.

10. The method of claim 1, wherein each of the electrical pulses are biphasic pulses.

11. The method of claim 1, wherein the one or more of the electrodes comprise at least three anodes and at least three cathodes.

12. The method of claim 11, wherein the anodes are configured as a first three or more adjacent electrodes and the cathodes are each configured as a second set of three or more adjacent electrodes.

13. The method of claim 12, wherein:
the anodes each share an anodic current fractionalized among each of the first three or more adjacent electrodes, and
the cathodes each share a cathodic current fractionalized among each of the second three or more adjacent electrodes.

14. The method of claim 13, wherein the anodic current is fractionalized equally among the first three or more adjacent electrodes and the cathodic current is fractionalized equally among the second three or more adjacent electrodes.

15. The method of claim 13, wherein:
the first set of adjacent electrodes comprises one or more rostral anodes, one or more middle anodes, and one or more caudal anodes,
the rostral anodes and the caudal anodes each share more of the anodic current than the middle anodes,
the second set of adjacent electrodes comprises one or more rostral cathodes, one or more middle cathodes and one or more caudal cathodes, and
the rostral cathodes and the caudal cathodes each share more cathodic current than the middle cathodes.

16. A system, comprising:
a spinal cord stimulator comprising:
stimulation circuitry programmed to generate a plurality of electrical stimulation pulses at a plurality of electrodes, wherein each of the pulses have a shape comprising an amplitude and a pulse width,
wherein each of the pulses are configured to be below the patient's perception threshold, and
wherein the amplitudes of the plurality of pulses are ramped from a first amplitude value to second amplitude value at a rate of no more than 3 mA/s.

17. The system of claim 16, wherein the pulse widths of the plurality of pulses vary over the first duration.

18. The system of claim 17, wherein the amplitudes of the plurality of pulses increase and the pulse widths of the plurality of pulses decrease over the first duration.

19. The system of claim 17, wherein the amplitudes of the plurality of pulses decrease and the pulse widths of the plurality of pulses increase over the first duration.

20. The system of claim 16, wherein the one or more of the electrodes comprise at least three anodes and at least three cathodes.
